(19) United States
(12) Patent Application Publication
(10) Pub. No.: US 2026/0047901 A1
(43) Pub. Date: Feb. 19, 2026

(54) CONTROL COMPONENT FOR ROBOTIC MICROSURGICAL PROCEDURES

(71) Applicant: FORSIGHT ROBOTICS LTD., Yokneam Illit (IL)

(72) Inventors: Zev SOHN, Karnei Shomron (IL); Daniel GLOZMAN, Kfar Yona (IL); Ori BEN ZEEV, Ramat Hasharon (IL); Tal KORMAN, Tel Aviv (IL); Muans OMARI, Sandala (IL)

(21) Appl. No.: 19/302,538

(22) Filed: Aug. 18, 2025

Related U.S. Application Data
(63) Continuation of application No. PCT/IB2024/051675, filed on Feb. 21, 2024.
(60) Provisional application No. 63/447,260, filed on Feb. 21, 2023.

(51) Int. Cl. A61B 34/30

(52) U.S. Cl. CPC A61B 34/30; A61B 2034/305

(57) ABSTRACT

Apparatus and methods are described including a control-component unit that includes a plurality of links that are coupled to each other via a plurality of axes. A control-component tool is coupled to the links and configured to be moved by an operator such that as the operator moves the control-component tool along linear X, Y, and Z directions, the links rotate around the linear-movement-related. An X-direction link through which X-direction linear motion is effected, is aligned with a Z-movement-related axis about which movement in the Z-direction is effected, such that the X-direction link does not exert any torque about the Z-movement-related axis. Other applications are also described.

### CROSS-REFERENCES TO RELATED APPLICATIONS

The present application is a continuation of PCT Application No. PCT/IB2024/051675 to Sohn (published as WO 2024/176143), filed Feb. 21, 2024, entitled “Control component for robotic microsurgical procedures,” which claims priority from U.S. Provisional Patent Application No. 63/447,260 to Sohn, filed Feb. 21, 2023, entitled “Control component for robotic microsurgical procedures,” which is incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention, a robotic system is configured for use in a microsurgical procedure, such as intraocular surgery. Typically, when used for intraocular surgery, the robotic system includes one or more robotic units (which are configured to hold tools), in addition to an imaging system, one or more displays and a control-component (e.g., a control-component that includes a pair of control component-units), via which one or more operators (e.g., healthcare professionals, such as a physician and/or a nurse) is able to control robotic units. Typically, the robotic system includes one or more computer processors, via which components of the system and operator(s) operatively interact with each other. The scope of the present application includes mounting one or more robotic units in any of a variety of different positions with respect to each other.

Typically, movement of the robotic units (and/or control of other aspects of the robotic system) is at least partially controlled by one or more operators (e.g., healthcare professionals, such as a physician and/or a nurse). For example, the operator may receive images of the patient's eye and the robotic units and/or tools disposed therein, via a display. Typically, such images are acquired by the imaging system. For some applications, the imaging system is a stereoscopic imaging device and display is a stereoscopic display. Based on the received images, the operator typically performs steps of the procedure. For some applications, the operator provides commands to the robotic units via the control-component. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), forceps, and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, the control component includes one or more control-component units that are configured to correspond to respective robotic units of the robotic system. For example, the system may include first and second robotic units, and the control component may include first and second control-component units. For some applications, the control-component units comprise respective control-component tools therein (in order to replicate the robotic units). Typically, the computer processor determines the XYZ location and orientation of a tip of the control-component tool, and drives the robotic unit such that the tip of the actual tool that is being used to perform the procedure tracks the movements of the tip of the control-component tool.

For some applications, in order to detect the XYZ location and three-dimensional orientation of a tip of the control-component tool, the control-component unit includes location sensors. The location sensors typically include one or more rotary encoders and/or one or more inertial-measurement units (which typically include a three-axis accelerometer, a three-axis gyroscope, and/or a three-axis magnetometer). The inertial-measurement unit typically generates inertial-measurement-unit data relating to a three-dimensional orientation of the control-component tool. For some applications, the computer processor receives the rotary-encoder data and/or the inertial-measurement-unit data, and thereby determines the XYZ location and three-dimensional orientation of the tip of the control-component tool.

For some applications, the control-component unit is configured to provide force feedback to the user. In order to perform non-robotic anterior ophthalmic surgery, a surgeon typically makes one or more incisions in the patient's cornea, which is thereafter used as an entry point for various surgical tools. A tool is inserted through an incision, and is manipulated within the eye to achieve the surgical goals. While this manipulation occurs, it is medically preferable that the tool does not forcefully press against the incision edges, lift upwards, or depress downwards exceedingly. Such motions may cause tearing at the incision edges, which widens the incision and can negatively impact the surgical outcome. Ideally, the surgeon will manipulate a tool such that at the entry point of the tool through the incision, the tool is rotated about the center of the incision and not moved laterally, with such motion of the tool at the incision being described herein as maintaining the center of motion. For robotic procedures, such as those described herein, the above-described motion of the ophthalmic tool is described as maintaining a remote center of motion, since the tool is typically controlled from a distance (via the control component). In non-robotic procedures, it can be difficult to manually maintain a center of motion, especially when the surgeon needs to focus on the tool tip, which is performing the current surgical action. In accordance with some applications of the present invention, force feedback is provided to assist an operator performing robotic-assisted ophthalmic surgery. The feedback, which is typically provided by the control component (as described in further detail hereinbelow), typically assists the operator in maintaining the remote center of motion of the ophthalmic tool.

For some applications, the computer processor is configured to drive the control-component unit to provide feedback (e.g., force feedback) to the operator that is indicative of a location of the entry of the tool into the patient's eye within the incision. For example, as the tool is moved in such a manner that the entry location of the tool into the patient's eye is closer to the edge of the incision, resistance to movement of the control-component arm may be increased, and/or the control-component arm may be vibrated, and/or a different output may be generated. For some applications, the computer processor is configured to apply forces that oppose the operator's attempted movements of the control-component tool that would result in violation of the remote center of motion. For some applications, in order to provide the above-described force feedback, the control-component unit includes one or more motors, as described in further detail hereinbelow. For some applications, at least some of the motors are direct-drive motors (i.e., motors that do not impart motion via gear wheels), and are typically linear motors, e.g., linear voice coil motors.

There is therefore provided, in accordance with some embodiments of the present invention, apparatus for use with a robotic unit configured to perform a procedure on a portion of a body of a patient using one or more tools, the apparatus including:
a control-component unit that includes:
   X, Y and Z linear-motion rotational axes, and pitch, roll and yaw angular-motion rotational axes; and
   a control-component tool coupled to the X, Y and Z linear-motion rotational axes and the pitch, roll and yaw angular-motion rotational axes and configured to be moved by an operator such that:
      as the operator moves the control-component tool along linear X, Y, and Z directions, rotational motion is generated about X, Y, and Z linear-motion rotational axes, and
      as the operator moves the control-component tool through roll, pitch and yaw angular motions, rotational motion is generated about respective pitch, roll and yaw angular-motion rotational axes,
   the control-component tool being substantially balanced around the X, Y and Z linear-motion rotational axes, and pitch, roll and yaw angular-motion rotational axes.

In some embodiments:
within four degrees of freedom, the control-component tool is self-balancing, and
within two degrees of freedom, the control-component unit includes counterweights such as to balance weight of the control-component and/or other components of the control-component unit about corresponding rotational axes.

In some embodiments, the control-component tool is self-balancing about the roll and yaw angular-motion rotational axes and about two of the X, Y, and Z linear-motion rotational axes, and the control-component includes first and second counterweights such as to balance weight of the control-component tool and/or other components of the control-component unit about the pitch angular-motion rotational axis and about one of the linear-motion rotational axes, respectively.

In some embodiments, the first counterweight does not entirely balance weight of the control-component tool about the pitch angular-motion rotational axis.

In some embodiments, the control-component tool is configured to maintain its position and orientation, in an absence of any forces acting upon the control-component tool.

In some embodiments, in response to the operator letting go of the control-component tool without exerting any forces on the control-component tool, the control-component tool is configured to maintain its position and orientation.

In some embodiments, the control-component unit includes one or more motors that are configured to provide force feedback to the operator by driving the control-component tool to move.

In some embodiments, the one or more motors are configured to provide the force feedback substantially without being required to overcome inertial forces.

In some embodiments, the one or more motors includes one or more direct-drive motors.

In some embodiments, the one or more motors includes one or more direct-drive linear motors.

In some embodiments, the one or more motors includes one or more direct-drive linear voice coil motors.

There is further provided, in accordance with some embodiments of the present invention, apparatus for use with a robotic unit configured to perform a procedure on a portion of a body of a patient using one or more tools, the apparatus including:
a control-component unit that includes:
   a plurality of links that are coupled to each other via a plurality of rotational axes; and
   a control-component tool coupled to the link and configured to be moved by an operator such that as the operator moves the control-component tool along linear X, Y, and Z directions, the links rotate around the rotational axes, and:
      the plurality of links include an X-direction link through which X-direction linear motion is effected,
      the plurality of rotational axes include a Z rotational axis about which movement in the Z-direction is effected, and
      the X-direction link is aligned with the Z rotational axis, such that the X-direction link does not exert any torque about the Z rotational axis.

In some embodiments, the plurality of rotational axes include a Y rotational axis about which movement in the Y direction is effected, and the Y rotational axis is aligned with the Z rotational axis along the Z direction.

In some embodiments, the X-direction link includes a frame.

In some embodiments, by virtue of the X-direction link being aligned with the Z rotational axis, the X-direction link does not exert any torque about the Z rotational axis.

In some embodiments, as the X-direction link undergoes motion, the X-direction link remains aligned with the Z rotational axis, such that no compensatory motion is necessary in order to balance the motion of the X-direction link.

In some embodiments, within four degrees of freedom, the control-component tool is self-balancing, and within two degrees of freedom, the control-component includes counterweights such as to balance weight of the control-component and/or other components of the control-component unit about corresponding rotational axes.

In some embodiments, the control-component tool is configured to maintain its position and orientation, in an absence of any forces acting upon the control-component tool.

In some embodiments, in response to the operator letting go of the control-component tool without exerting any forces on the control-component tool, the control-component tool is configured to maintain its position and orientation.

In some embodiments, the control-component unit includes one or more motors that are configured to provide force feedback to the operator by driving the control-component tool to move.

In some embodiments, the one or more motors are configured to provide the force feedback substantially without being required to overcome inertial forces.

In some embodiments, the one or more motors includes one or more direct-drive motors.

In some embodiments, the one or more motors includes one or more direct-drive linear motors.

In some embodiments, the one or more motors includes one or more direct-drive linear voice coil motors.

There is further provided, in accordance with some embodiments of the present invention, apparatus for use with a robotic unit configured to perform a procedure on a portion of a body of a patient using one or more tools, the apparatus including:
a control-component unit that includes:
   X, Y and Z linear-motion rotational axes and pitch, roll and yaw angular-motion rotational axes; and
   a control-component tool coupled to the X, Y and Z linear-motion rotational axes and the pitch, roll and yaw angular-motion rotational axes and configured to be moved by an operator such that:
      as the operator moves the control-component tool along linear X, Y, and Z directions, rotational motion is generated about X, Y, and Z linear-motion rotational axes, and
      as the operator moves the control-component tool through roll, pitch and yaw angular motions, rotational motion is generated about respective pitch, roll and yaw angular-motion rotational axes;
   a plurality of direct-drive motors that are operatively coupled to respective X, Y, and Z linear-motion rotational axes; and
a computer processor configured to:
   move the tip of the selected ophthalmic tool within the patient's eye in a manner that corresponds with movement of the control-component tool; and
   provide force feedback to the operator by driving the control-component arm using the plurality of direct-drive motors.

In some embodiments, the plurality of direct-drive motors includes a plurality of linear motors.

In some embodiments, the plurality of linear motors include a plurality of linear voice coil motors.

In some embodiments, the direct drive motors are configured to avoid motor cogging.

In some embodiments, the direct drive motors are configured to provide force feedback to the operator that is more accurate than the force feedback that would be provided by motors that undergo motor clogging.

There is further provided, in accordance with some embodiments of the present invention, apparatus for use with a robotic unit configured to perform a procedure on a portion of a body of a patient using one or more tools, the apparatus including:
a control-component unit that includes:
   X, Y and Z linear-motion rotational axes and pitch, roll and yaw angular-motion rotational axes; and
   a control-component tool coupled to the X, Y and Z linear-motion rotational axes and the pitch, roll and yaw angular-motion rotational axes and configured to be moved by an operator such that:
      as the operator moves the control-component tool along linear X, Y, and Z directions, rotational motion is generated about X, Y, and Z linear-motion rotational axes, and
      as the operator moves the control-component tool through roll, pitch and yaw angular motions, rotational motion is generated about respective pitch, roll and yaw angular-motion rotational axes;
   X-direction, Y-direction, and Z-direction motors that are operatively coupled respectively to X, Y, and Z linear-motion rotational axes; and a computer processor configured to:
   move the tip of the selected ophthalmic tool within the patient's eye in a manner that corresponds with movement of the control-component tool; and
   provide force feedback to the operator by driving the control-component arm using the X-direction, Y-direction, and Z-direction motors,
   a first end of the Y-direction motor being aligned with the X rotational axis.

In some embodiments, the control component includes a frame and the control-component unit is configured such that as the operator moves the control-component tool along the linear X, direction, it causes the frame to rotate about the X linear-motion rotational axis, and the Y-direction motor is coupled to the frame such that the Y-direction motor rotes with the frame.

In some embodiments, when the Y-direction motor extends or contracts, it does not exert any torque about the X rotational axis.

In some embodiments, a second end of the Y-direction motor is offset from the Y rotational axis such that when the Y-direction motor extends or contracts, it exerts a torque about the Y rotational axis.

In some embodiments, the second end of the Y-direction motor is offset from the Y rotational axis by between 5 mm and 20 mm.

In some embodiments, the second end of the Y-direction motor is offset from the Y rotational axis by between 5 mm and 20 mm.

In some embodiments, the second end of the Y-direction motor is offset from the Y rotational axis by between 10 mm and 15 mm.

In some embodiments, the X-direction, Y-direction, and Z-direction motors include direct drive motors.

In some embodiments, the direct-drive motors include linear motors.

In some embodiments, the linear motors include linear voice coil motors.

In some embodiments, the direct drive motors are configured to avoid motor cogging.

In some embodiments, the direct drive motors are configured to provide force feedback to the operator that is more accurate than the force feedback that would be provided by motors that undergo motor clogging.

In some embodiments, a center-of-mass of the Y-direction motor is substantially aligned with the X linear-motion rotational axis.

In some embodiments, the Y-direction motor includes a linear motor and both when the Y-direction motor is fully extended and fully retracted, its center of mass is within 10 mm of the X linear-motion rotational axis.

In some embodiments, the Y-direction motor includes a linear motor and both when the Y-direction motor is fully extended and fully retracted, its center of mass is within 5 mm of the X linear-motion rotational axis.

There is further provided, in accordance with some embodiments of the present invention, apparatus for use with a robotic unit configured to perform a procedure on a portion of a body of a patient using one or more tools, the apparatus including:
a control-component unit that includes:
   X, Y and Z linear-motion rotational axes and a pitch, roll and yaw angular-motion rotational axes; and
   a control-component tool coupled to the X, Y and Z linear-motion rotational axes and the pitch, roll and yaw angular-motion rotational axes and configured to be moved by an operator such that:
      as the operator moves the control-component tool along linear X, Y, and Z directions, rotational motion is generated about X, Y, and Z linear-motion rotational axes, and
      as the operator moves the control-component tool through roll, pitch and yaw angular motions, rotational motion is generated about respective pitch, roll and yaw angular-motion rotational axes;
   at least one rotary encoder that is configured to detect rotational motion about a corresponding one of the rotational axes;
   one or more wires extending from the rotary encoder; and
   a toroidal magnet disposed along the corresponding one of the rotational axes, the one or more wires passing through a hole defined by the toroidal magnet.

In some embodiments, control-component unit includes:
a plurality of rotary encoders each of which is configured to detect rotational motion about a respective one of the X, Y, and Z linear-motion rotational axes, and each of which has one or more wires extending therefrom, and
toroidal magnets disposed along of the X, Y, and Z linear-motion rotational axes, the one or more wires passing through holes defined by the toroidal magnets.

In some embodiments, the control-component unit includes:
a plurality of rotary encoders each of which is configured to detect rotational motion about a respective one of the pitch, roll and yaw angular-motion rotational axes, and each of which has one or more wires extending therefrom, and
toroidal magnets disposed along of the pitch, roll and yaw angular-motion rotational axes, the one or more wires passing through holes defined by the toroidal magnets.

In some embodiments, the control-component unit further includes:
a plurality of rotary encoders each of which is configured to detect rotational motion about a respective one of the X, Y, and Z linear-motion rotational axes, and each of which has one or more wires extending therefrom, and
toroidal magnets disposed along of the X, Y, and Z linear-motion rotational axes, the one or more wires passing through holes defined by the toroidal magnets.

The present invention will be more fully understood from the following detailed description of embodiments thereof, taken together with the drawings, in which:

### BRIEF DESCRIPTION OF THE DRAWINGS

FIG. 1 is a schematic illustration of a robotic system that is configured for use in a microsurgical procedure, such as intraocular surgery, in accordance with some applications of the present invention;

FIGS. 2A and 2B are schematic illustrations of respective views of a control-component unit, in accordance with some applications of the present invention.

FIGS. 3A, 3B, and 3C are schematic illustrations of portions of a control-component unit, in accordance with some applications of the present invention.

FIGS. 4A and 4B are schematic illustrations of movement of a control-component tool in X and Y linear directions, in accordance with some applications of the present invention;

FIG. 4C is a schematic illustration of a Y-direction motor of a control-component unit, in accordance with some applications of the present invention;

FIGS. 5A and 5B are schematic illustrations of movement of a control-component tool in the Z linear direction, in accordance with some applications of the present invention;

FIGS. 6A and 6B are schematic illustrations of pitch angular motion of a control-component tool, in accordance with some applications of the present invention;

FIGS. 7A and 7B are schematic illustrations of yaw angular motion of a control-component tool, in accordance with some applications of the present invention;

FIG. 8 is a schematic illustration of a portion of a control-component tool, in accordance with some applications of the present invention; and

FIGS. 9A, 9B, 9C, and 9D are schematic illustrations of a control-component unit, in accordance with some applications of the present invention.

### DETAILED DESCRIPTION OF EMBODIMENTS

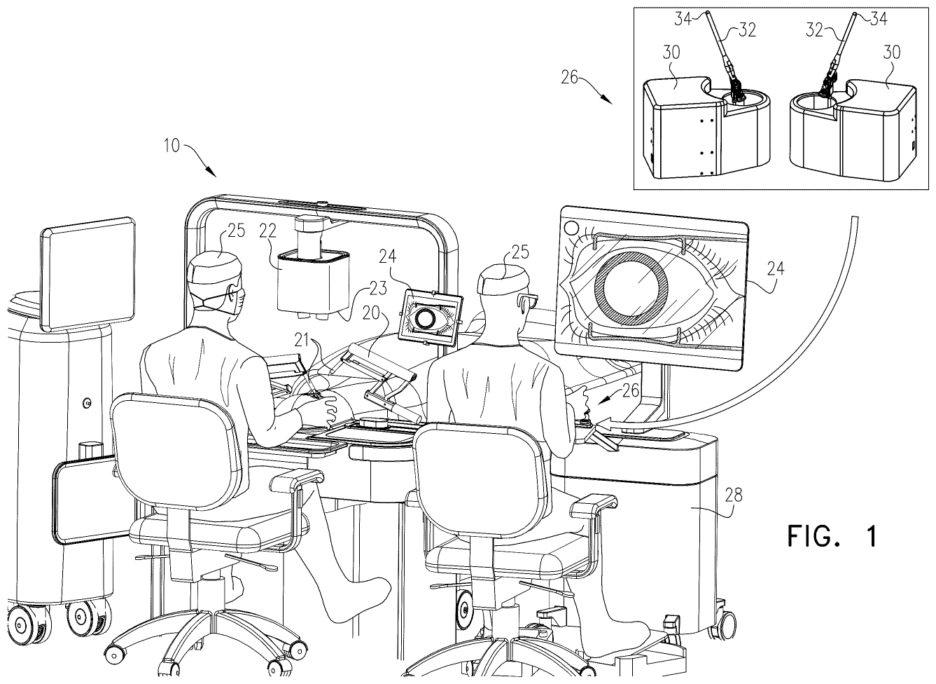

Reference is now made to FIG. 1, which is a schematic illustration of a robotic system 10 that is configured for use in a microsurgical procedure, such as intraocular surgery, in accordance with some applications of the present invention. Typically, when used for intraocular surgery, robotic system 10 includes one or more robotic units 20 (which are configured to hold tools 21), in addition to an imaging system 22, one or more displays 24 and a control component 26 (e.g., a control component that includes a pair of control component-units 30, as shown in the enlarged portion of FIG. 1), via which one or more operators 25 (e.g., healthcare professionals, such as a physician and/or a nurse) is able to control robotic units 20. Typically, robotic system 10 includes one or more computer processors 28, via which components of the system and operator(s) 25 operatively interact with each other. The scope of the present application includes mounting one or more robotic units in any of a variety of different positions with respect to each other.

Typically, movement of the robotic units (and/or control of other aspects of the robotic system) is at least partially controlled by one or more operators 25 (e.g., healthcare professionals, such as a physician and/or a nurse). For example, the operator may receive images of the patient's eye and the robotic units and/or tools disposed therein, via display 24. Typically, such images are acquired by imaging system 22. For some applications, imaging system 22 is a stereoscopic imaging device and display 24 is a stereoscopic display. Based on the received images, the operator typically performs steps of the procedure. For some applications, the operator provides commands to the robotic units via control component 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), forceps, and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye.

Typically, control component 26 includes one or more control-component units 30 that are configured to correspond to respective robotic units 20 of the robotic system. For example, as shown, the system may include first and second robotic units, and the control component may include first and second control-component units 30, as shown. For some applications, the control-component units comprise respective control-component tools 32 therein (in order to replicate the robotic units), as shown in FIG. 1. Typically, the computer processor determines the XYZ location and orientation of a tip 34 of the control-component tool 32, and drives the robotic unit such that the tip of the actual tool 21 that is being used to perform the procedure tracks the movements of the tip of the control-component tool. In some cases, tool 21 is described herein, in the specification and in the claims, as an “ophthalmic tool.” This term is used in order to distinguish tool 21 from control-component tool 32, and should not be interpreted as limiting the type of tool that may be used as tool 21 in any way. The term “ophthalmic tool” should be interpreted to include any one the tools described herein and or any other types of tools that may occur to a person of ordinary skill in the art upon reading the present disclosure.

For some applications, in order to detect the XYZ location and three-dimensional orientation of a tip 34 of the control-component tool 32, the control-component unit includes location sensors. The location sensors typically include one or more rotary encoders and/or one or more inertial-measurement units (which typically include a three-axis accelerometer, a three-axis gyroscope, and/or a three-axis magnetometer). The inertial-measurement unit typically generates inertial-measurement-unit data relating to a three-dimensional orientation of the control-component tool. For some applications, computer processor 28 receives the rotary-encoder data and/or the inertial-measurement-unit data, and thereby determines the XYZ location and three-dimensional orientation of the tip of the control-component tool.

For some applications, control-component unit 30 is configured to provide force feedback to the user. In order to perform non-robotic anterior ophthalmic surgery, a surgeon typically makes one or more incisions in the patient's cornea, which is thereafter used as an entry point for various surgical tools. A tool is inserted through the incision, and is manipulated within the eye to achieve the surgical goals. While this manipulation occurs, it is medically preferable that the tool does not forcefully press against the incision edges, lift upwards, or depress downwards exceedingly. Such motions may cause tearing at the incision edges, which widens the incision and can negatively impact the surgical outcome. Ideally, the surgeon will manipulate a tool such that at the entry point of the tool through the incision, the tool is rotated about the center of the incision and not moved laterally, with such motion of the tool at the incision being described herein as maintaining the center of motion. For robotic procedures, such as those described herein, the above-described motion of tool 21 is described as maintaining a remote center of motion, since the tool is typically controlled from a distance (via control-component unit 30). In non-robotic procedures, it can be difficult to manually maintain a center of motion, especially when the surgeon needs to focus on the tool tip, which is performing the current surgical action. In accordance with some applications of the present invention, force feedback is provided to assist an operator performing robotic-assisted ophthalmic surgery. The feedback, which is typically provided by control-component unit 30 (as described in further detail hereinbelow), typically assists the operator in maintaining the remote center of motion of tool 21.

For some applications, the computer processor is configured to drive the control-component unit to provide feedback to the operator that is indicative of a location of the entry of the tool into the patient's eye within the incision. For example, as the tool is moved in such a manner that the entry location of the tool into the patient's eye is closer to the edge of the incision, resistance to movement of the control-component arm may be increased, and/or the control-component arm may be vibrated, and/or a different output may be generated. For some applications, the computer processor is configured to apply forces that oppose the operator's attempted movements of control-component tool 32 that would result in violation of the remote center of motion. For some applications, in order to provide the above-described force feedback, the control-component unit includes one or more motors, as described in further detail hereinbelow. For some applications, at least some of the motors are direct-drive motors (i.e., motors that do not impart motion via gear wheels), and are typically linear motors, e.g., linear voice coil motors.

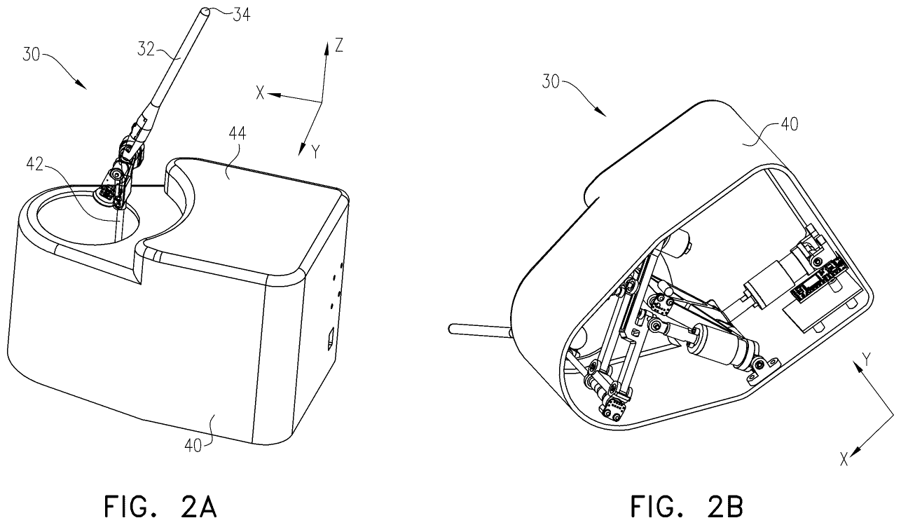

Reference is now made to FIGS. 2A and 2B, which are schematic illustrations of respective views of control-component unit 30, in accordance with some applications of the present invention. For some applications, portions of the control-component unit are housed within a housing 40. Typically, control-component tool 32 is disposed outside the housing such that it is moveable by the operator. For example, a shaft 42 may extend outside of housing 40, with the control-component tool mounted on the shaft. For some applications, the housing is shaped such as to define a surface 44 that is configured to support the palm and/or heel of the operator's hand while they are operating the control-component tool. As described hereinabove, typically, the computer processor determines the XYZ location and orientation of tip 34 of the control-component tool 32, and drives the robotic unit such that the tip of the ophthalmic tool tracks the movements of the tip of the control-component tool.

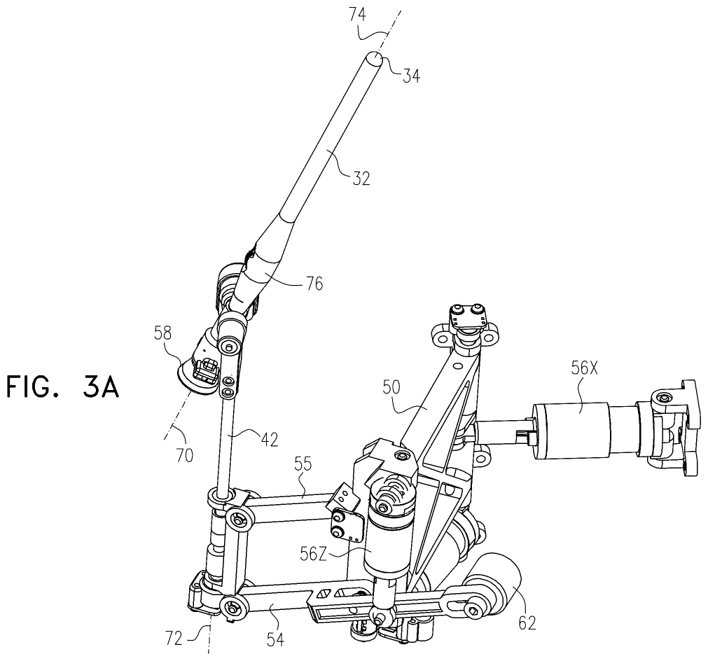

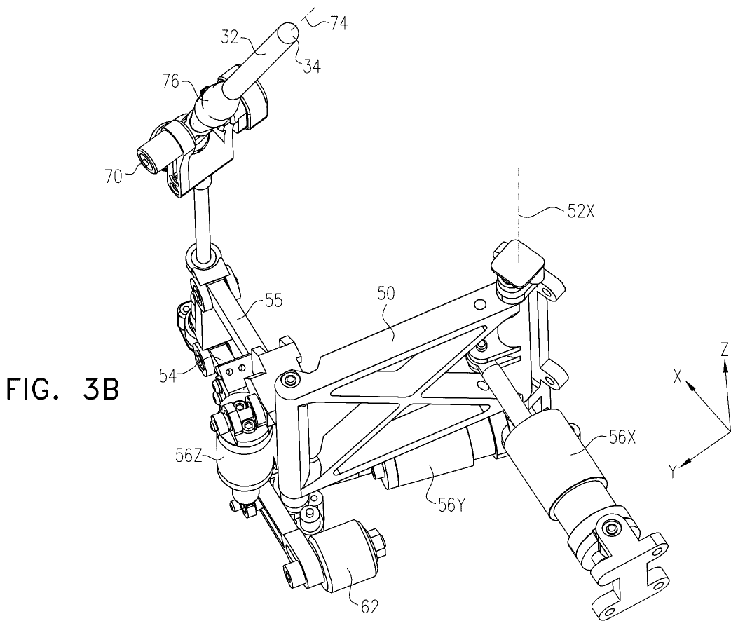

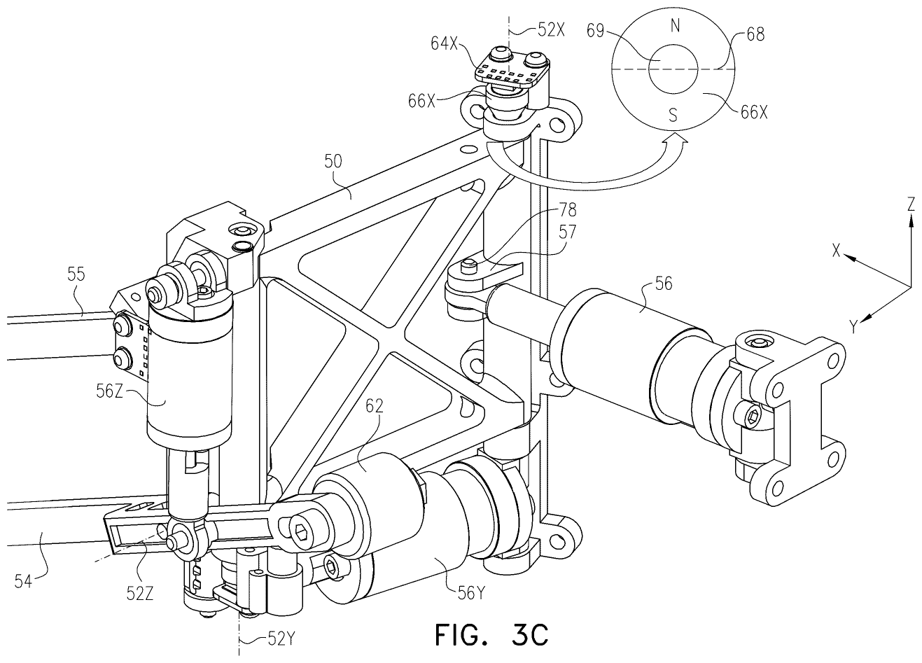

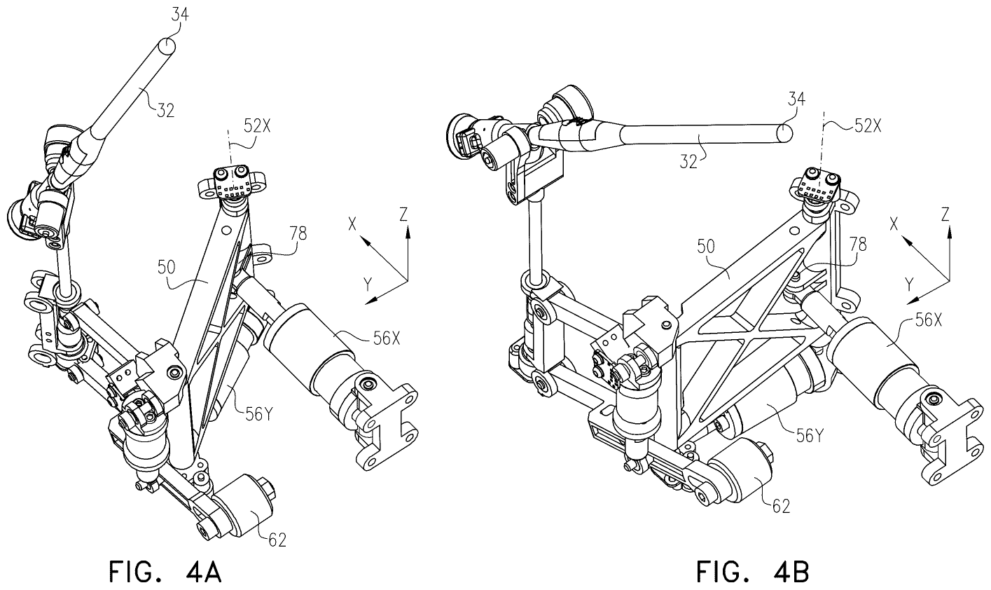

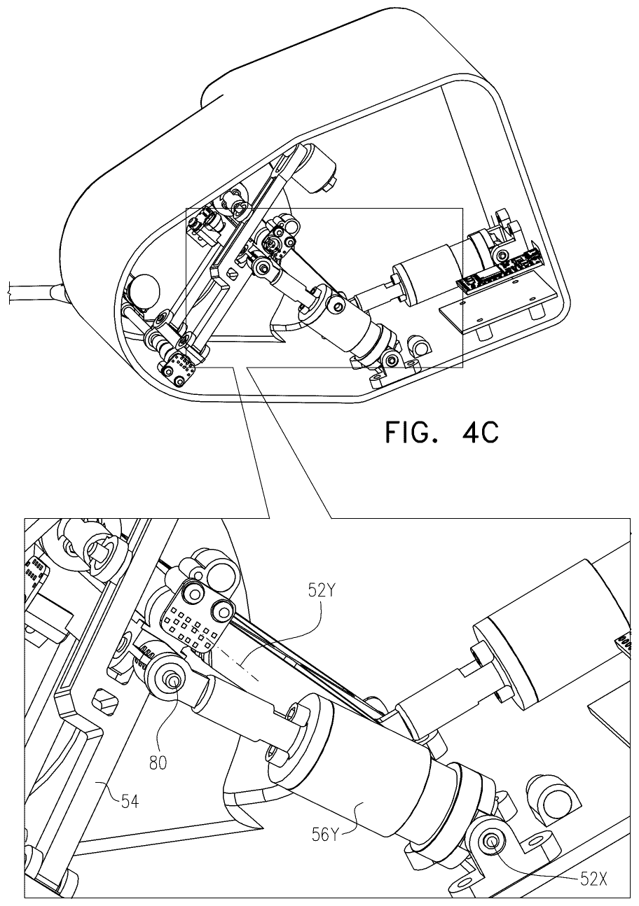

Reference is now made to FIGS. 3A, 3B, and 3C, which are schematic illustrations of portions of control-component unit 30, in accordance with some applications of the present invention. For some applications, the control-component unit includes a plurality of links (at least some of which are typically configured as frames), the links being coupled to each other via rotational axes. Typically, as the operator moves the control-component tool along X, Y, and Z linear direction, this causes links to rotate around respective rotational axes. For example, as the operator moves the control-component tool along the X linear direction, this causes frame 50 to rotate about rotational axis 52X (shown in FIGS. 3B and 3C), as the operator moves the control-component tool along the Y linear direction, this causes link 54 to rotate about rotational axis 52Y (shown in FIGS. 3C and 4C), and as the operator moves the control-component tool along the Z linear direction, this causes link 54 to rotate about rotational axis 52Z (shown in FIG. 3C).

It is noted that the above description assumes that link 54 is disposed perpendicularly to frame 50. In practice, during much of the use of the control-component unit, link 54 is disposed at an angle to frame 50 (e.g., as shown in FIG. 4A). In such configurations, movement of the control-component tool within the X-Y plane (even along the X linear direction or along the Y linear direction) will typically result in both frame 50 rotating about rotational axis 52X and link 54 rotating about rotational axis 52Y. For this reason, the use of the terms X, Y, and Z as used herein in relation to movements of portions of the control-component unit should not be interpreted as strictly corresponding to movement along three linear axes that are perpendicular to each other. Rather, movement in the X and Y directions should be interpreted as relating to movement of frame 50 or link 54 within an X-Y plane (but not necessarily in directions that are perpendicular to each other) and movement in the Z direction should be interpreted as corresponding to movement of link 54 in a direction that is perpendicular to the X-Y plane. Thus, rotational axis 52X and motor 56X are associated with movement of frame 50 within the X-Y plane (regardless of whether the movement is in the X direction as indicated in the figures), rotational axis 52Y and motor 56Y are associated with movement of link within the X-Y plane (regardless of whether the movement is in the Y direction as indicated in the figures), and rotational axis 52Z and motor 56Z are associated with movement of link 54 perpendicularly to the X-Y plane.

Typically, as shown, the Y rotational axis 52Y is aligned with Z rotational axis 52Z along the Z direction. Further typically, both Y and Z linear motion are effected via link 54. It is noted that, for some applications, an additional supporting link 55 is disposed parallel to link 54 and rotates together with link 54. For some applications, a rotary encoder is disposed along each of the rotational axes 52X, 52Y, and 52Z (or a parallel rotational axis (e.g., the rotational axis of link 55). The rotary encoders detect rotation of respective links about the rotational axes, and generates signals in response thereto. The computer processor derives motion of the control-component tool along respective linear directions from the signal generated by the rotary encoders. For some applications, at least one additional rotary encoder is disposed along each of the rotational axes 52X, 52Y, and 52Z in order to provide the system with redundancy (e.g., such that in the event that one of the rotary encoders malfunctions, the other rotary encoder is used).

Typically, control-component tool 32 is moveable by the operator to undergo pitch, yaw, and roll angular rotations. The control-component tool typically undergoes pitch angular rotation by rotating about pitch rotational axis 70 (shown in FIG. 3A), and undergoes yaw angular rotation by shaft 42 (upon which the control-component tool is mounted) rotating about its own longitudinal axis 72 (which functions as the yaw rotational axis, and which is shown in FIG. 3A). Typically, the control-component tool undergoes roll angular rotation by rotating about its own axis 74 (which functions as the roll rotational axis, and is shown in FIG. 3A). For some applications, an inertial-measurement unit 76 is housed within the control-component tool. Typically, the inertial measurement unit includes a three-axis accelerometer, a three-axis gyroscope, and/or a three-axis magnetometer. The inertial-measurement unit typically generates inertial-measurement-unit data relating to a three-dimensional orientation of the control-component tool. Alternatively or additionally, the control component includes one or more rotary encoders to detect the roll, pitch and/or yaw orientation of control-component tool 32. Typically, the rotary encoders are disposed along the axis about which the roll, pitch and yaw angular rotations occur, respectively. For some applications, the control component includes inertial-measurement unit 76 in addition to one or more rotary encoders to detect the roll, pitch and/or yaw of control-component tool 32, for redundancy (e.g., such that in the event that the inertial measurement unit malfunctions, the rotary encoders are used).

For some applications, computer processor 28 receives the rotary-encoder data and the inertial-measurement-unit data. Typically, the computer processor determines the XYZ location of the tip of the control-component tool 32 based upon the rotary-encoder data, and determines the three-dimensional orientation of the tip of control-component tool 32 (e.g., the 3 Euler angles of orientation, and/or another representation of orientation) based upon the inertial-measurement-unit data, or based upon a combination of the rotary-encoder data and the inertial-measurement-unit data. Thus, based upon the combination of the rotary-encoder data and the inertial-measurement-unit data, the computer processor is configured to determine the XYZ location and three-dimensional orientation of the tip of the control-component tool.

For some applications, a direct-drive motor 56X, 56Y, 56Z (i.e., motors that do not impart motion via gear wheels), which is typically a linear motor (e.g., a linear voice coil motor), is associated with motion along each of the X, Y, and Z linear directions. As described hereinabove, for some applications, the computer processor is configured to drive the control-component unit to provide force feedback to the operator that is indicative of a location of the entry of the ophthalmic tool into the patient's eye within the incision. For some applications, the motors are configured to drive the tool to move linearly, in order to provide the aforementioned force feedback. For some applications, the computer processor is configured to apply forces that oppose the operator's attempted movements of control-component tool 32 that would result in violation of the remote center of motion. For example, in response to the operator moving the control-component tool through an angular yaw rotation that would cause a corresponding movement of the ophthalmic tool that would violate the remote center of motion, the computer processor may move the control-component tool linearly (through X, Y, and/or X linear motion) such that the remote center of motion of the ophthalmic tool is maintained. For some such applications, the forces are applied by driving the control-component tool to move in the X, Y, and Z linear directions, via motors 56X, 56Y, 56Z.

Typically, robotic system 10 is used in procedures that require delicate and precise movements of the surgical tools, e.g., ophthalmic procedures, as described hereinabove. Therefore, control-component unit 30 is typically configured such that movement of control-component tool is performed by the operator without there being substantial counterforces to the movement (other than counterforces that are deliberately applied via motors 56X, 56Y, 56Z). For some applications, the control-component tool includes a counterweight 58, such that the weight of the control-component tool is relatively evenly balanced about pitch rotational axis 70. For some applications, the control-component tool is not entirely balanced about pitch rotational axis 70, in order to give the physician a feeling of the tool's weight (like a real surgical tool), and/or also to reduce the overall mass of the control-component tool. For some applications, link 54 extends across both sides of the Z rotational axis 52Z, with the control-component tool and additional components being disposed on link 54 (and/or parallel link 55) on a first side of rotational axis 52Z. For some applications, a counterweight 62 is disposed on link 54 on the other side of rotational axis 52Z, such as to balance the weight of the control-component tool and additional components that are disposed on the first side. For some applications, frame 50 (which functions as the link through which X direction linear motion is effected) is aligned with Z rotational axis 52Z (shown in FIG. 3C), such that frame 50 does not exert any torque about Z rotational axis 52Z. Thus, frame 50 does not need to be counterbalanced about Z rotational axis 52Z. For some applications, even as frame 50 moves (due to motion in the X direction), the frame remains aligned with Z rotational axis 52Z, such that no compensatory motion is necessary in order to balance the motion of the frame.

It is noted that in accordance with the above description, the control-component unit typically is balanced within all six degrees of freedom (the three axial translations and three angular rotations). For some applications, as described, the control-component unit utilizes counterweights to provide balance in two degrees of freedom: Z direction axial motion and pitch angular motion. The remaining four degrees of freedom (i.e., X and Y axial motion, and roll and yaw angular motions) typically do not require counterweights for balancing, since the control-component unit is designed such that the control-component tool and/or other elements of the control-component unit are self-balancing within these degrees of freedom. Since the control-component unit is designed to be balanced within all six degrees of freedom (e.g., by self-balancing within four degrees of freedom and balance being provided by the counterweights within the two remaining degrees of freedom), the control-component tool tends to maintain its position and orientation, in the absence of any forces acting upon the control-component tool. Thus, typically if the operator temporarily lets go of the control-component tool (without exerting force on the control-component tool as she/he lets go of the tool), the control-component tool maintains its position and orientation until the operator resumes control of the control-component tool. Further typically, the control-component tool is able to provide force feedback to the operator at relatively low levels of force, since the control-component tool provides relatively low inertial forces. I.e., the motors that are configured to provide force feedback to the operator by driving the control-component tool to move are configured to do so substantially without being required to overcome inertial forces.

As described hereinabove, typically, direct-drive motors (i.e., motors that do not impart motion via gear wheels) are used for motors 56X, 56Y, 56Z. For some applications, linear motors (and typically, linear voice coil motors) are used for motors 56X, 56Y, 56Z. For some applications, such motors are used for motors 56X, 56Y, 56Z, in order to avoid motor cogging, which can provide resistance to motion of the control-component tool (and which is common with rotary motors and/or motors that impart motion via gear wheels). It is noted that motor cogging could also lead to the force feedback that is provided by the movement of the motors being inaccurate, which is typically avoided by using direct-drive motors (and typically linear motors, such as linear voice coil motors) for motors 56X, 56Y, 56Z. Alternatively, for some applications, motors that include gear wheels are used for one or more of motors 56X, 56Y, and 56Z.

Referring to FIG. 3C, as noted above, for some applications, a rotary encoder is disposed along each of the rotational axes 52X, 52Y, and 52Z. The rotary encoders detect rotation of respective links about the rotational axes, and generates signals in response thereto. In FIG. 3C, rotary encoder 64X of the X rotational axis 52X is shown. Typically, a magnet 66X is disposed along the X rotational axis. For some applications, the magnet is toroidal with the North and South poles of the magnet being on opposite sides of a line 68 that bisects the toroidal shape, as schematically illustrated in FIG. 3C. Rotary encoder 64X detects changes in the magnetic flux that is generated by magnet 66X and thereby detects rotation of X rotational axis 52X. Typically, the magnet is toroidal such that electrical wires (e.g., wires extending from rotary encoder 64X) pass through a hole 69 defined by the magnet. In this manner the wires remain stationary even as the magnet rotates, thereby avoiding the wires becoming twisted. The inventors have found that even with the magnet having the toroidal shape, the magnetic flux that is generated by the magnet is sufficiently strong to be detected by the rotary encoder. It is noted that although rotary encoder 64X of the X rotational axis 52X is shown in and described with reference to FIG. 3C, typically additional rotary encoders are configured in a similar manner (e.g., using a toroidal magnet as described). Typically, similarly configured rotary encoders (and toroidal magnets) are used to detect rotation about the Y rotational axis, the Z rotational axis, the yaw rotational axis, the pitch rotational axis, and/or the roll rotational axis.

Reference is now made to FIGS. 4A and 4B, which are schematic illustrations of movement of a control-component tool in X and Y linear directions, in accordance with some applications of the present invention. As described hereinabove, typically, as the operator moves the control-component tool within the X-Y plane, this causes links to rotate around respective rotational axes. For example, as the operator moves the control-component tool along the X linear direction, this causes frame 50 to rotate about rotational axis 52X (shown in FIGS. 3B and 3C), as the operator moves the control-component tool along the Y linear direction, this causes link 54 to rotate about rotational axis 52Y (shown in FIG. 4C). It is noted that, for some applications, an additional support link 55 is disposed parallel to link 54 and rotates together with link 54. Movement within the X-Y plane is shown in the transition from 4A to FIG. 4B, with FIG. 4A showing both frame 50 and link 54 in contracted configurations, and FIG. 4B showing both frame 50 and link 54 in extended configurations. For some applications, a rotary encoder is disposed along rotational axes 52X and 52Y. The rotary encoders detect rotation of respective links about the rotational axes, and generates signals in response thereto. The computer processor derives motion of the control-component tool within the X-Y plane from the signal generated by the rotary encoders. For some applications, at least one additional rotary encoder is disposed along each of the rotational axes 52X and 52Y in order to provide the system with redundancy (e.g., such that in the event that one of the rotary encoders malfunctions, the other rotary encoder is used).

Also as described hereinabove, for some applications, a motor 56X, 56Y is disposed along each of the X and Y linear directions. Typically, each of the motors is a direct-drive motor, e.g., a direct-drive linear motor, such as a linear voice coil motor. For some applications, the motors are configured to drive the tool to move within the X-Y plane, in order to provide force feedback. For some applications, the computer processor is configured to apply forces that oppose the operator's attempted movements of control-component tool 32 that would result in violation of the remote center of motion. For example, in response to the operator moving the control-component tool through an angular yaw or pitch rotation that would cause a corresponding movement of the ophthalmic tool that would violate the remote center of motion, the computer processor may move the control-component tool linearly (through X, Y, and/or Z linear motion) such that the remote center of motion of the ophthalmic tool is maintained. For some such applications, the forces are applied by driving the control-component tool to move within the X-Y plane, via motors 56X and 56Y.

Referring to FIGS. 4A and 4B, typically, the X-direction motor 56X (or a linear extension therefrom) is coupled to frame 50 at a location 78 that is offset from the X rotational axis 52X. For some applications, location 78 is offset from X rotational axis 52X by between 3 mm and 30 mm, e.g., between 5 and 20 mm, e.g., between 10 and 15 mm. Thus, when the X-direction motor 56X extends or contracts, it exerts torque about the X rotational axis (thereby causing movement of the control-component tool within the X-Y plane). For some applications, motor 56X (or a linear extension therefrom) is coupled to frame 50 at location 78, which is located on an extension 57 of frame 50, which is disposed within the footprint of the frame, as shown in FIG. 3C.

Reference is now made to FIG. 4C, which is a schematic illustration of Y-direction motor 56Y of control-component unit 30, in accordance with some applications of the present invention. As shown for some applications, a first end of the Y-direction motor (or a linear extension therefrom) is aligned with the X rotational axis 52X. Typically, the second end of the Y-direction motor (or a linear extension therefrom) is coupled to link 54 at a location 80 that is offset from the Y rotational axis 52Y. For some applications, location 80 is offset from Y rotational axis 52Y by between 3 mm and 30 mm, e.g., between 5 and 20 mm, e.g., between 10 and 15 mm. Thus, when the Y-direction motor 56Y extends or contracts, it does not exert any torque about the X rotational axis 52X (which would require a compensatory torque and/or motion in the X direction), but it does exert torque about the Y rotational axis (thereby causing movement of the control-component tool within the X-Y plane). Typically, motor 56Y is coupled to frame 50, such that motor 56Y is configured to rotate together with frame 50. By being configured in this manner, the motor does not apply any torque to frame 50 even as frame 50 rotates.

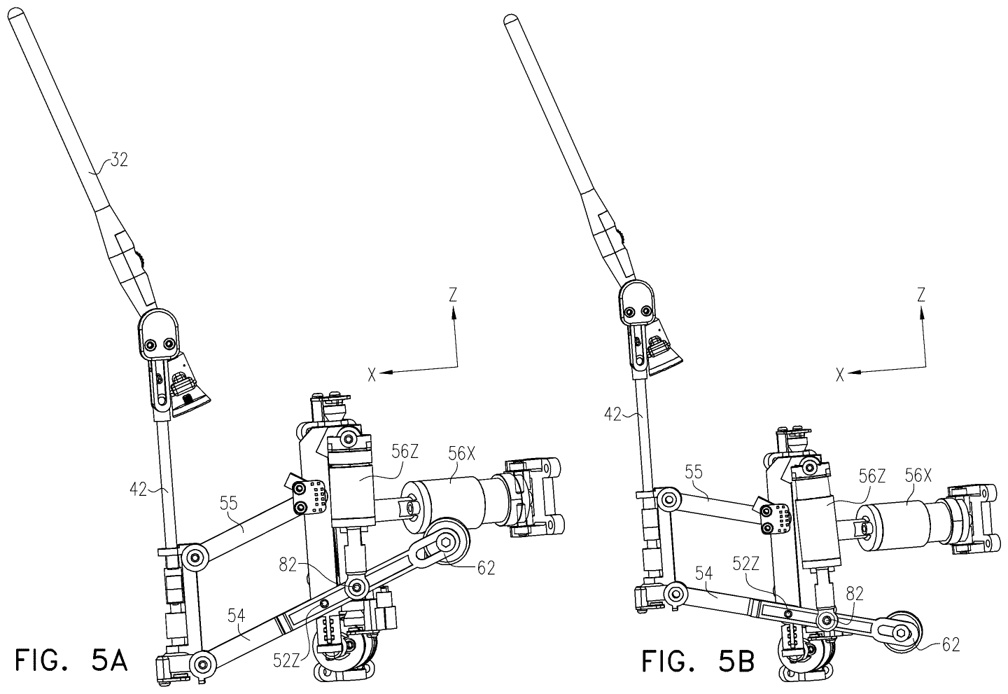

Reference is now made to FIGS. 5A and 5B, which are schematic illustrations of movement of a control-component tool in the Z linear direction, in accordance with some applications of the present invention. As described hereinabove, typically, as the operator moves the control-component tool along the Z linear direction, this causes link 54 to rotate around Z rotational axis 52Z. It is noted that, for some applications, an additional support link 55 is disposed parallel to link 54 and rotates together with link 54. Movement along the Z direction is shown in the transition from 5A to FIG. 5B, with FIG. 5A showing link 54 in a contracted configuration (along the Z direction), and FIG. 5B showing link 54 in it extended configuration (along the Z direction). For some applications, a rotary encoder is disposed along rotational axis 52Z (or a parallel rotational axis, e.g., a rotational axis passing through link 55). The rotary encoder detects rotation of link 54 about rotational axis 52Z, and generates signals in response thereto. The computer processor derives motion of the control-component tool along the Z direction from the signal generated by the rotary encoder. For some applications, at least one additional rotary encoder is disposed along the Z rotational axis (or a parallel rotational axis, e.g., a rotational axis passing through link 55) in order to provide the system with redundancy (e.g., such that in the event that one of the rotary encoders malfunctions, the other rotary encoder is used).

Also as described hereinabove, for some applications, a motor 56Z is disposed along the Z linear direction. Typically, the motor is a direct-drive motor, e.g., a direct-drive linear motor, such as a linear voice coil motor. For some applications, the motor is configured to drive the tool to move along the Z linear direction, in order to provide force feedback. For some applications, the computer processor is configured to apply forces that oppose the operator's attempted movements of control-component tool 32 that would result in violation of the remote center of motion. For example, in response to the operator moving the control-component tool through an angular yaw or pitch rotation that would cause a corresponding movement of the ophthalmic tool that would violate the remote center of motion, the computer processor may move the control-component tool linearly (through X, Y, and/or Z linear motion) such that the remote center of motion of the ophthalmic tool is maintained. For some such applications, the forces are applied by driving the control-component tool to move in the Z linear direction, via motor 56Z. As shown, the motor is coupled to link 54 at a location 82 that is offset from Z rotational axis 52Z. For some applications, location 82 is offset from Z rotational axis 52Z by between 3 mm and 30 mm, e.g., between 5 and 20 mm, e.g., between 10 and 15 mm. Thus, when the Z-direction motor 56Z extends or contracts, it exerts torque about the Z rotational axis (thereby causing movement of the control-component tool along the Z direction).

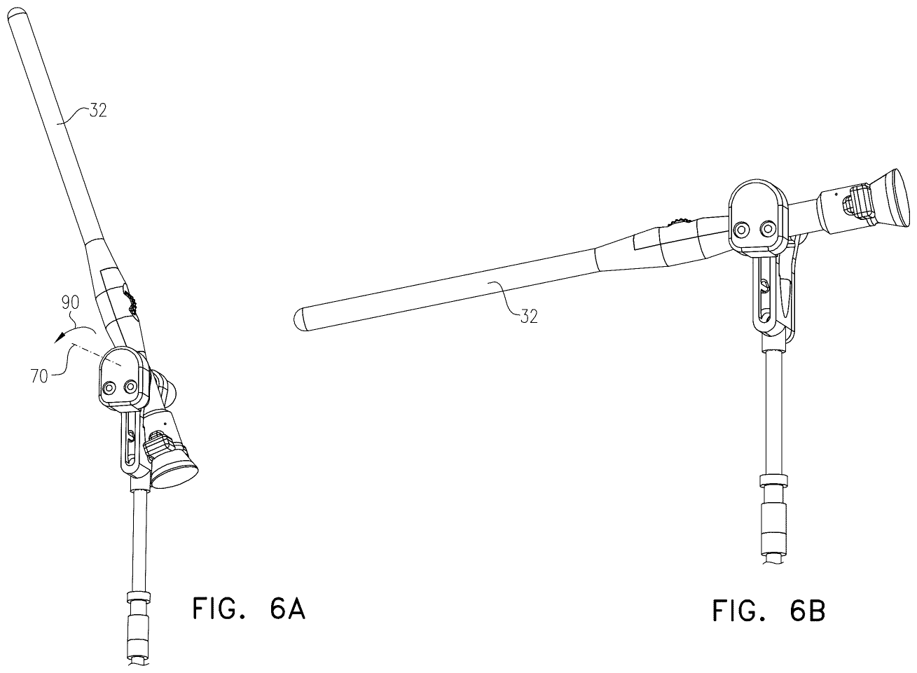

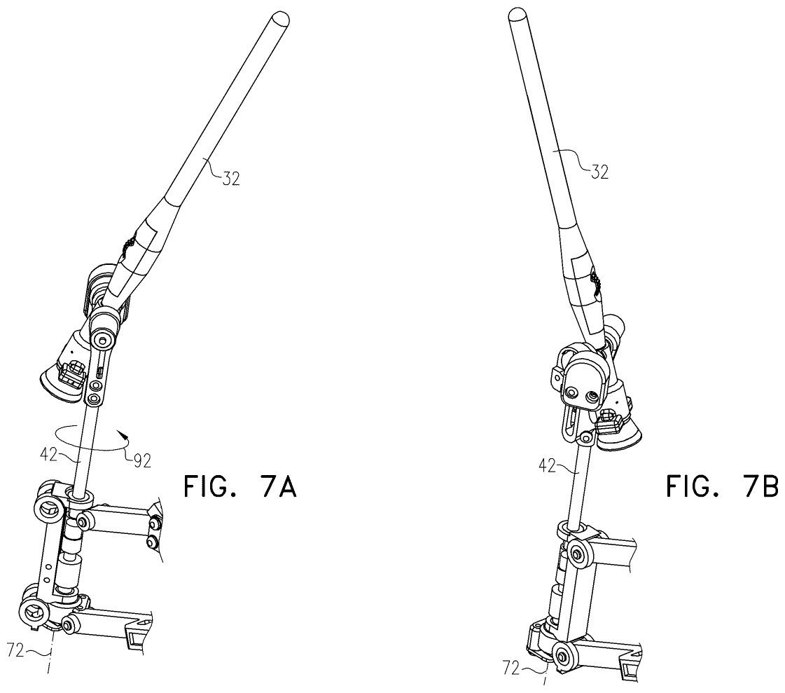

Reference is now made to FIGS. 6A and 6B, which are schematic illustrations of pitch angular motion of control-component tool 32, in accordance with some applications of the present invention. Reference is also made to FIGS. 7A and 7B, which are schematic illustrations of yaw angular motion of a control-component tool, in accordance with some applications of the present invention. As described hereinabove, the control-component tool typically undergoes pitch angular rotation by rotating about pitch rotational axis 70. This is illustrated in the transition from FIG. 6A to FIG. 6B, with the pitch rotation being indicated by arrow 90. Further typically, the tool undergoes yaw angular rotation by shaft 42 (upon which the control-component tool is mounted) rotating about its own axis 72 (which functions as the yaw rotational axis). This is illustrated in the transition from FIG. 7A to FIG. 7B, with the rotation of the shaft being indicated by arrow 92.

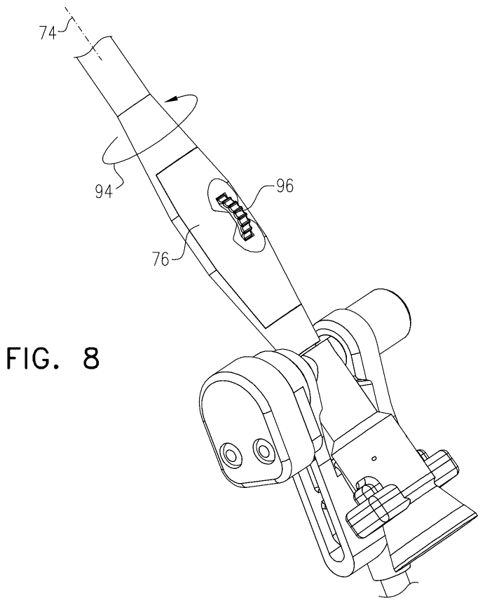

Reference is now made to FIG. 8, which is a schematic illustration of a portion of a control-component tool, in accordance with some applications of the present invention. As described hereinabove, typically, the control-component tool undergoes roll angular rotation by rotating about its own axis 74, as indicated by arrow 94.

Referring to the angular rotations that are schematically illustrated in FIGS. 6A-8, as described hereinabove, for some applications, an inertial-measurement unit 76 is housed within the control-component tool. Typically, the inertial measurement unit includes a three-axis accelerometer, a three-axis gyroscope, and/or a three-axis magnetometer. The inertial-measurement unit typically generates inertial-measurement-unit data relating to a three-dimensional orientation of the control-component tool. Alternatively or additionally, the control component includes one or more rotary encoders to detect the roll, pitch and/or yaw orientation of control-component tool 32. Typically, the rotary encoders are disposed along the axis about which the roll, pitch and yaw angular rotations occur, respectively. For some applications, the control component includes inertial-measurement unit 76 in addition to one or more rotary encoders to detect the roll, pitch and/or yaw of control-component tool 32, for redundancy (e.g., such that in the event that the inertial measurement unit malfunctions, the rotary encoders are used).

As described hereinabove, for some applications, the operator provides commands to the robotic units via control component 26. Typically, such commands include commands that control actions that are performed by the tools. For example, the commands may control a blade, a phacoemulsification tool (e.g., the operation mode and/or suction power of the phacoemulsification tool), forceps, and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, and/or x-y positioning of the imaging system). For some applications, the commands include controlling an intraocular-lens-manipulator tool, for example, such that the tool manipulates the intraocular lens inside the eye for precise positioning of the intraocular lens within the eye. For some applications, the control-component tool (an/or a different portion of the control-component unit) includes one or more components that are configured to receive such inputs from the operator. For example, as shown in FIG. 8, the control-component tool may include a roller wheel 96. Alternatively or additionally, the control-component tool may include a different type of component that is configured to receive such inputs from the operator, such as buttons, etc.

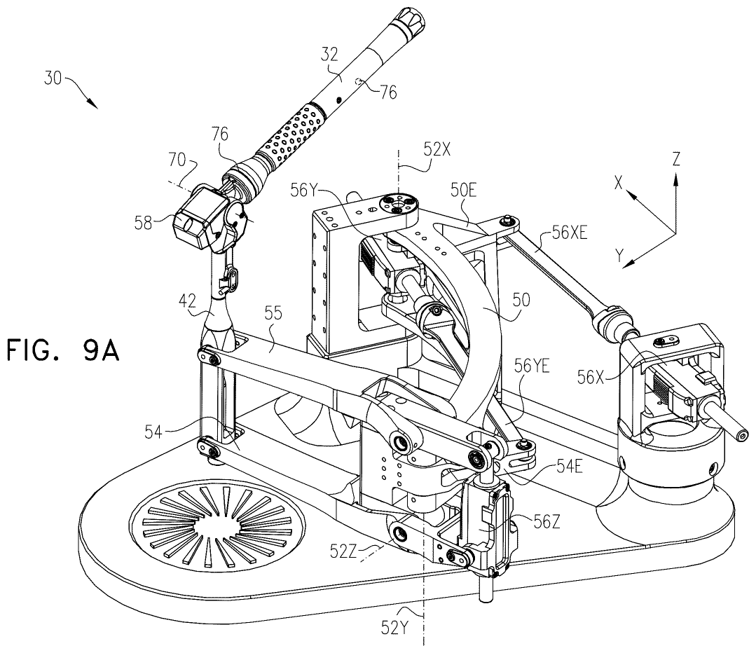

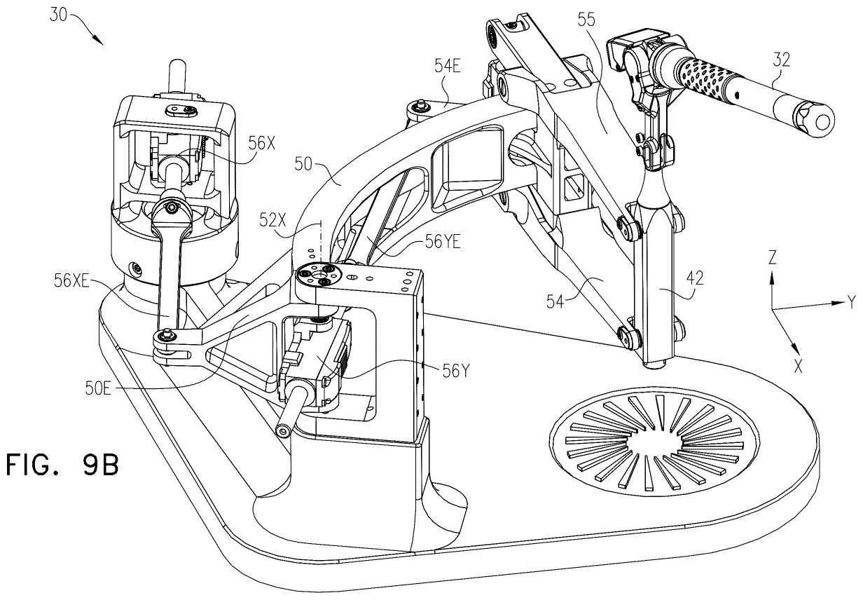

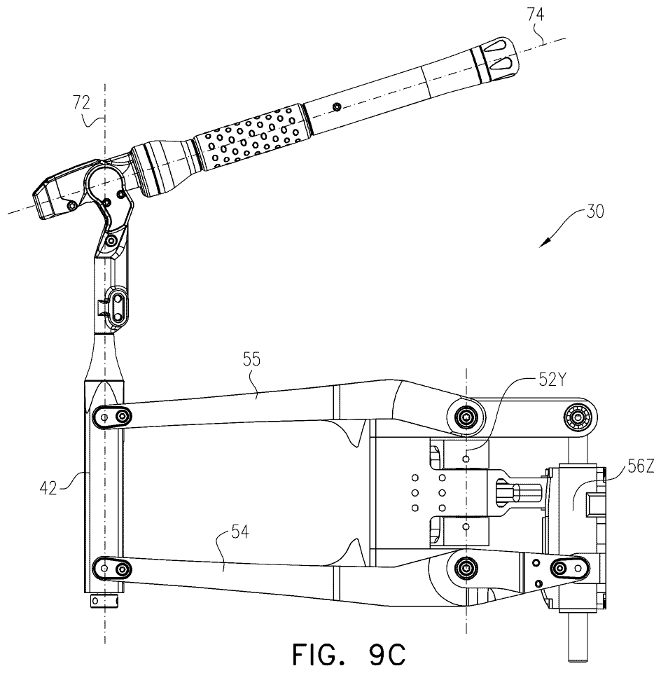

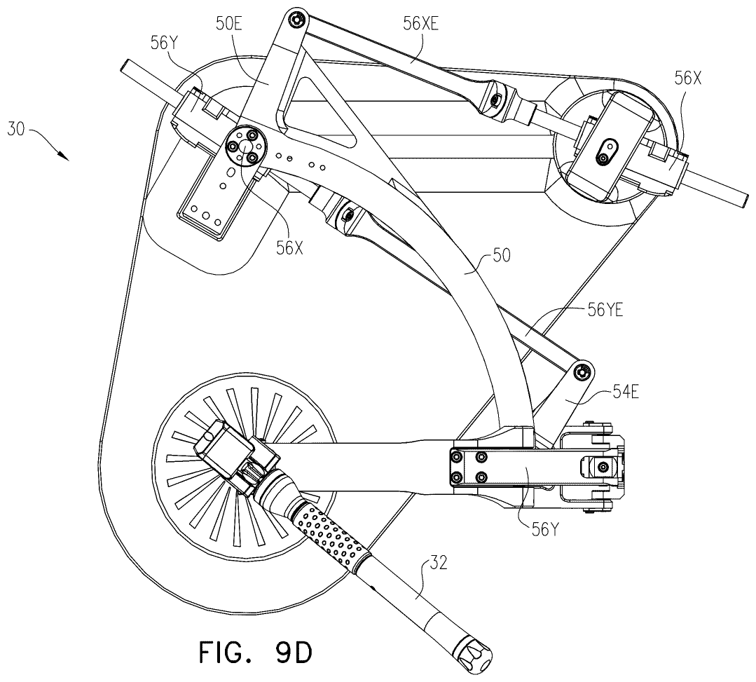

Reference is now made to FIGS. 9A, 9B, 9C, and 9D, which are schematic illustrations of control-component unit 30, in accordance with some applications of the present invention. FIGS. 9A and 9B show respective oblique views of the control-component unit, FIG. 9C shows a side view, and FIG. 9D shows a top view. The structure and functionality of control-component unit 30 as shown in FIGS. 9A-9B is generally similar to that of control-component unit 30 as shown in FIGS. 2A-8, except for the differences described hereinbelow.

Control-component unit 30 as shown in FIGS. 9A-9B is generally similar to that of control-component unit 30 as shown in FIGS. 2A-8 in the several respects. The control-component unit typically includes frame 50, which rotates around a first rotational axis 52X, and a link 54, which rotates around a second rotational axis 52Y and around a third rotational axis 52Z. Typically, the operator moving the control-component tool along X, Y, and Z linear directions causes links to rotate around respective rotational axes. For example, as the operator moves the control-component tool along the X linear direction, this causes frame 50 to rotate about rotational axis 52X, as the operator moves the control-component tool along the Y linear direction, this causes link 54 to rotate about rotational axis 52Y (shown in FIGS. 3C and 4C), and as the operator moves the control-component tool along the Z linear direction, this causes link 54 to rotate about rotational axis 52Z (shown in FIG. 3C).

As noted above, the above description assumes that link 54 is disposed perpendicularly to frame 50. In practice, during much of the use of the control-component unit, link 54 is disposed at an angle to frame 50 (e.g., as shown in FIG. 4A). In such configurations, movement of the control-component tool within the X-Y plane (even along the X linear direction or along the Y linear direction) will typically result in both frame 50 rotating about rotational axis 52X and link 54 rotating about rotational axis 52Y. For this reason, the use of the terms X, Y, and Z as used herein in relation to movements of portions of the control-component unit should not be interpreted as strictly corresponding to movement three linear axes that are perpendicular to each other. Rather, movement in the X and Y directions should be interpreted as relating to movement of frame 50 or link 54 within an X-Y plane (but not necessarily in directions that are perpendicular to each other) and movement in the Z direction should be interpreted as corresponding to movement of link 54 in a direction that is perpendicular to the X-Y plane. Thus, rotational axis 52X and motor 56X are associated with movement of frame 50 within the X-Y plane (regardless of whether the movement is in the X direction as indicated in the figures), rotational axis 52Y and motor 56Y are associated with movement of link within the X-Y plane (regardless of whether the movement is in the Y direction as indicated in the figures), and rotational axis 52Z and motor 56Z are associated with movement of link 54 perpendicularly to the X-Y plane.

Typically, as shown, the Y rotational axis 52Y is aligned with Z rotational axis 52Z along the Z direction. Further typically, both Y and Z linear motion are effected via link 54. It is noted that, for some applications, an additional supporting link 55 is disposed parallel to link 54 and rotates together with link 54. For some applications, link 54 and/or link 55 are made of two or more portions that are rigidly to each other. For example, as shown in FIG. 9A, links 54 and 55 each includes a first portion disposed to the left of Z rotational axis 52Z, and a second portion disposed to the right of Z rotational axis 52Z. For some applications, a rotary encoder is disposed along each of the rotational axes 52X, 52Y, and 52Z (or a parallel rotational axis (e.g., the rotational axis of link 55). The rotary encoders detect rotation of respective links about the rotational axes, and generates signals in response thereto. The computer processor derives motion of the control-component tool along respective linear directions from the signal generated by the rotary encoders. For some applications, at least one additional rotary encoder is disposed along each of the rotational axes 52X, 52Y, and 52Z in order to provide the system with redundancy (e.g., such that in the event that one of the rotary encoders malfunctions, the other rotary encoder is used).

As described with reference to control-component unit 30 as shown in FIGS. 2A-8, typically, control-component tool 32 is moveable by the operator to undergo pitch, yaw, and roll angular rotations. The control-component tool typically undergoes pitch angular rotation by rotating about pitch rotational axis 70, and undergoes yaw angular rotation by shaft 42 (upon which the control-component tool is mounted) rotating about its own axis 72 (which functions as the yaw rotational axis). Typically, the control-component tool undergoes roll angular rotation by rotating about its own axis 74 (which functions as the roll rotational axis). For some applications, an inertial-measurement unit 76 is housed within the control-component tool. Typically, the inertial measurement unit includes a three-axis accelerometer, a three-axis gyroscope, and/or a three-axis magnetometer. The inertial-measurement unit typically generates inertial-measurement-unit data relating to a three-dimensional orientation of the control-component tool. Alternatively or additionally, the control component includes one or more rotary encoders to detect the roll, pitch and/or yaw orientation of control-component tool 32. Typically, the rotary encoders are disposed along the axis about which the roll, pitch and yaw angular rotations occur, respectively. For some applications, the control component includes inertial-measurement unit 76 in addition to one or more rotary encoders to detect the roll, pitch and/or yaw of control-component tool 32, for redundancy (e.g., such that in the event that the inertial measurement unit malfunctions, the rotary encoders are used).

As described with reference to control-component unit 30 as shown in FIGS. 2A-8, typically, computer processor 28 receives the rotary-encoder data and the inertial-measurement-unit data. Typically, the computer processor determines the XYZ location of the tip of the control-component tool 32 based upon the rotary-encoder data, and determines the three-dimensional orientation of the tip of control-component tool 32 (e.g., the 3 Euler angles of orientation, and/or another representation of orientation) based upon the inertial-measurement-unit data, or based upon a combination of the rotary-encoder data and the inertial-measurement-unit data. Thus, based upon the combination of the rotary-encoder data and the inertial-measurement-unit data, the computer processor is configured to determine the XYZ location and three-dimensional orientation of the tip of the control-component tool.

As described with reference to control-component unit 30 as shown in FIGS. 2A-8, typically, a direct-drive motor 56X, 56Y, 56Z (i.e., a motor that does not impart motion via gear wheels), and which is typically a linear motor (e.g., a linear voice coil motor)), is associated with motion along the X, Y, and Z linear directions. As described hereinabove, for some applications, the computer processor is configured to drive the control-component unit to provide force feedback to the operator that is indicative of a location of the entry of the ophthalmic tool into the patient's eye within the incision. For some applications, the motors are configured to drive the tool to move linearly, in order to provide the aforementioned force feedback. For some applications, the computer processor is configured to apply forces that oppose the operator's attempted movements of control-component tool 32 that would result in violation of the remote center of motion. For example, in response to the operator moving the control-component tool through an angular yaw rotation that would cause a corresponding movement of the ophthalmic tool that would violate the remote center of motion, the computer processor may move the control-component tool linearly (through X, Y, and/or X linear motion) such that the remote center of motion of the ophthalmic tool is maintained. For some such applications, the forces are applied by driving the control-component tool to move in the X, Y, and Z linear directions, via motors 56X, 56Y, 56Z.

Typically, robotic system 10 is used in procedures that require delicate and precise movements of the surgical tools, e.g., ophthalmic procedures, as described hereinabove. Therefore, control-component unit 30 is typically configured such that movement of control-component tool is performed by the operator without there being substantial counterforces to the movement (other than counterforces that are deliberately applied via motors 56X, 56Y, 56Z). For some applications, the control-component tool includes a counterweight 58, such that the weight of the control-component tool is relatively evenly balanced about pitch rotational axis 70. For some applications, the control-component tool is not entirely balanced about pitch rotational axis 70, in order to give the physician a feeling of the tool's weight (like a real surgical tool), and/or also to reduce the overall mass of the control-component tool. For some applications, link 54 extends across both sides of the Z rotational axis 52Z, with the control-component tool and additional components being disposed on link 54 (and/or parallel link 55) on a first side of rotational axis 52Z. For some applications, motor 56Z, which is disposed along the Z linear direction is disposed on link 54 on the other side of rotational axis 52Z, such as to balance the weight of the control-component tool and additional components that are disposed on the first side. For some such application, the control-component unit does not include an additional counterweight for this purpose (unlike control-component unit as shown in FIGS. 2A-8, which includes dedicate counterweight 62 for this purpose). Alternatively, the control-component unit include a counterweight for this purpose, in addition to motor 56Z.

For some applications, frame 50 (which functions as the link through which X direction linear motion is effected) comprises two curved arms and motor 56Y (and, optionally, an extension 56YE thereof) passes between the two curved arms along a straight line. For some applications, an end of frame 50 which is adjacent to Z rotational axis 52Z is aligned with Z rotational axis 52Z (as shown in FIG. 9A), such that frame 50 does not exert any torque about Z rotational axis 52Z. Thus, frame 50 does not need to be counterbalanced about Z rotational axis 52Z. For some applications, even as frame 50 moves (due to motion in the X direction), the frame remains aligned with Z rotational axis 52Z, such that no compensatory motion is necessary in order to balance the motion of the frame.

It is noted that in accordance with the above description, the control-component unit typically is balanced within all six degrees of freedom (the three axial translations and three angular rotations). For some applications, as described, the control-component unit utilizes counterweights to provide balance in two degrees of freedom: Z direction axial motion and pitch angular motion. (In the embodiment shown in FIGS. 9A-9D, motor 56Z functions as the counterweight in the Z direction axial motion degree of freedom). The remaining four degrees of freedom (i.e., X and Y axial motion, and roll and yaw angular motions) typically do not require counterweights for balancing, since the control-component unit is designed such that the control-component tool and/or other elements of the control-component unit are self-balancing within these degrees of freedom. Since the control-component unit is designed to be balanced within all six degrees of freedom (e.g., by self-balancing within four degrees of freedom and balance being provided by the counterweights within the two remaining degrees of freedom), the control-component tool tends to maintain its position and orientation, in the absence of any forces acting upon the control-component tool. Thus, typically if the operator temporarily lets go of the control-component tool (without exerting force on the control-component tool as she/he lets go of the tool), the control-component tool maintains its position and orientation until the operator resumes control of the control-component tool. Further typically, the control-component tool is able to provide force feedback to the operator at relatively low levels of force, since the control-component tool provides relatively low inertial forces. I.e., the motors that are configured to provide force feedback to the operator by driving the control-component tool to move are configured to do so substantially without being required to overcome inertial forces.

In addition to some differences described hereinabove, the structure and functionality of control-component unit 30 as shown in FIGS. 9A-9B differs from that of control-component unit 30 as shown in FIGS. 2A-8, in the following ways.

For some applications, as shown in FIGS. 9A-9B, motor 56Y is disposed within the X-Y plane such that its center of mass is substantially aligned with X rotational axis 52X both when motor 56Y is extended and when motor 56Y retracted. Typically, this prevents movement of motor 56Y from exerting any torque in the Z direction on link 54 as motor 56Y extends and contracts. It is noted that as the motor extends and contracts, its center of mass moves slightly. Typically, the motor is positioned such that in at least one position within its fully extended and fully contracted states, the motor's center of mass is aligned with X rotational axis 52X. Further typically, the motor's center of mass is aligned with X rotational axis 52X, when the motor is at its central position with respect to its fully extended and fully retracted states. For some applications, both when the motor is fully extended and fully retracted, its center of mass is within 10 mm, e.g., within 5 mm, of X rotational axis 52X. It is further noted that as with control-component unit as described with respect to FIGS. 2A-8, motor 56Y is coupled to frame 50, such that motor 56Y is configured to rotate together with frame 50. By being configured in this manner, the motor does not apply any torque to frame 50 even as frame 50 rotates.

For some applications, frame 50 includes an angled extension 50E to which motor 56X (and, optionally, an extension 56XE thereof) is coupled. Motor 56X rotates frame 50 about axis 52X by the motor (or the extension thereof) pushing or pulling angled extension 50E. Typically, by the control-component unit incorporating angled extension 50E, the dimensions of the control-component unit (and the overall footprint of the control component) are reduced relative to if motor 56X (or extension 56XE thereof) were to be coupled to non-angled continuation of frame 50 on an opposite side of axis 52X from the main portion of frame 50. For some applications, motor 56X rotates frame 50 about axis 52X by the motor (or the extension thereof) by pushing or pulling a non-angled extension 57 that is disposed within the footprint of the frame, e.g., as shown in FIG. 3B.

Similarly, for some applications, link 54 includes an angled extension 54E to which motor 56Y (and, optionally, an extension 56YE thereof) is coupled. Motor 56Y rotates link 54 about axis 52Y by the motor (or the extension thereof) pushing or pulling angled extension 54E. Typically, by the control-component unit incorporating angled extension 54E, the dimensions of the control-component unit (and the overall footprint of the control component) are reduced relative to if motor 56Y (or extension 56YE thereof) were to be coupled to non-angled continuation of link 54 on an opposite side of axis 52Y from the main portion of link 54. For some applications, motor 56Y rotates frame 50 about axis 52Y by the motor (or the extension thereof) by pushing or pulling link 54 at location 80 that is offset from the Y rotational axis 52Y, e.g., as shown in FIG. 4C.

For some applications, longitudinal axis 72 of shaft 42 (which functions as the yaw rotational axis) is aligned with the ends of links 54 and 55. This is in contrast to the embodiment shown in FIGS. 2A-8, in which shaft 42 is supported within an extension from links 54 and 56. Typically, by aligning longitudinal axis 72 of shaft 42 with the ends of parallel links 54 and 55, the dimensions of the control-component unit (and the overall footprint of the control component) are reduced relative to if shaft 42 is supported within an extension from links 54 and 56.

Although some applications of the present invention are described with reference to cataract surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to intraocular procedures, other than cataract surgery, mutatis mutandis. Such procedures may include collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for use with a robotic unit configured to perform a procedure on a portion of a body of a patient using one or more tools, the apparatus comprising:
a control-component unit that comprises:
a plurality of links that are coupled to each other via a plurality of axes that comprise linear-movement-related axes; and
a control-component tool coupled to the link and configured to be moved by an operator such that as the operator moves the control-component tool along linear X, Y, and Z directions, the links rotate about the linear-movement-related axes,
wherein:
the plurality of links comprise an X-direction link through which X-direction linear motion is effected,
the plurality of linear-movement-related axes comprise a Z-movement-related axis about which movement in the Z-direction is effected, and
the X-direction link is aligned with the Z-movement-related axis, such that the X-direction link does not exert any torque about the Z-movement-related axis.

2. The apparatus according to claim 1, wherein the plurality of linear-movement-related axes comprise a Y-movement-related axis about which movement in the Y direction is effected, and wherein the Y-movement-related axis is aligned with the Z-movement-related axis along the Z direction.

3. The apparatus according to claim 2, wherein the plurality of links further comprise a Y-direction link through which the movement in the Y direction is effected, wherein the X-direction link and Y-direction link are both disposed within an X-Y plane but are not always perpendicular to each other, and wherein the movement in the Z-direction is movement is a direction that is perpendicular to the X-Y plane.

4. The apparatus according to claim 1, wherein the X-direction link comprises a frame.

5. The apparatus according to claim 1, wherein by virtue of the X-direction link being aligned with the Z-movement-related axis, the X-direction link does not exert any torque about the Z-movement-related axis.

6. The apparatus according to claim 1, wherein as the X-direction link undergoes motion, the X-direction link remains aligned with the Z-movement-related axis, such that no compensatory motion is necessary in order to balance the motion of the X-direction link.

7. The apparatus according to claim 1, wherein within four degrees of freedom, the control-component tool is self-balancing, and within two degrees of freedom, the control-component comprises counterweights such as to balance weight of the control-component and/or other components of the control-component unit about corresponding axes.

8. The apparatus according to claim 1, wherein the control-component unit comprises one or more motors that are configured to provide force feedback to the operator by driving the control-component tool to move.

9. The apparatus according to claim 8, wherein the one or more motors comprises one or more direct-drive motors.

10. The apparatus according to claim 9, wherein the one or more motors comprises one or more direct-drive linear motors.

11. The apparatus according to claim 10, wherein the one or more motors comprises one or more direct-drive linear voice coil motors.

12. Apparatus for use with a robotic unit configured to perform a procedure on a portion of a body of a patient using one or more tools, the apparatus comprising:
a control-component unit that comprises:
X-movement-related, Y-movement-related, and Z-movement-related linear-movement-related joints and angular-movement-related joints; and
a control-component tool coupled to the linear-movement-related joints and the pitch, roll and yaw angular-movement-related joints and configured to be moved by an operator such that:
as the operator moves the control-component tool along linear X, Y, and Z directions, motion is generated about the linear-movement-related joints, and
as the operator moves the control-component tool through roll, pitch and yaw angular motions, motion is generated about the angular-movement-related joints;
a plurality of direct-drive motors that are operatively coupled respectively to the X-movement-related, Y-movement-related, and Z-movement-related joints; and
a computer processor configured to:
move the tip of one of the tools with respect to the portion of the patient's body in a manner that corresponds with movement of the control-component tool; and
provide force feedback to the operator by driving the control-component arm using the plurality of direct-drive motors.

13. The apparatus according to claim 12, wherein the plurality of direct-drive motors comprises a plurality of linear motors.

14. The apparatus according to claim 13, wherein the plurality of linear motors comprise a plurality of linear voice coil motors.

15. The apparatus according to claim 12, wherein the direct drive motors are configured to avoid motor cogging.

16. The apparatus according to claim 15, wherein the direct drive motors are configured to provide force feedback to the operator that is more accurate than the force feedback that would be provided by motors that undergo motor clogging.

17. An apparatus for use with a robotic unit configured to perform a procedure on a portion of a body of a patient using one or more tools, the apparatus comprising:
a control-component unit that comprises:
X-movement-related, Y-movement-related, and Z-movement-related linear-movement-related axes and angular-movement-related axes; and
a control-component tool coupled to the linear-movement-related axes and the angular-movement-related axes and configured to be moved by an operator such that:
as the operator moves the control-component tool along linear X, Y, and Z directions, rotational motion is generated about the linear-movement-related axes, and
as the operator moves the control-component tool through roll, pitch and yaw angular motions, rotational motion is generated about the angular-movement-related axes;
X-direction, Y-direction, and Z-direction motors that are operatively coupled respectively to the X-movement-related, Y-movement-related, and Z-movement-related axes; and
a computer processor configured to:
move the tip of one of the tools with respect to the portions of the patient's body in a manner that corresponds with movement of the control-component tool; and
provide force feedback to the operator by driving the control-component arm using the X-direction, Y-direction, and Z-direction motors,
wherein a first end of the Y-direction motor is aligned with the X-movement-related axis.

18. The apparatus according to claim 17, wherein the control component comprises a frame and the control-component unit is configured such that as the operator moves the control-component tool along the X direction, it causes the frame to rotate about the X-movement-related axis, and wherein the Y-direction motor is coupled to the frame such that the Y-direction motor rotates with the frame.

19. The apparatus according to claim 17, wherein when the Y-direction motor extends or contracts, it does not exert any torque about the X-movement-related axis.

20. The apparatus according to claim 17, wherein the X-direction, Y-direction, and Z-direction motors comprise direct drive motors.

21. The apparatus according to claim 17, wherein a center-of-mass of the Y-direction motor is substantially aligned with the X-movement-related axis.
